(19) United States
(12) Patent Application Publication
(10) Pub. No.: US 2017/0367606 A1
(43) Pub. Date: Dec. 28, 2017

(54) REHABILITATION SYSTEM USING ARTIFICIALLY INTELLIGENT HORSEBACK-RIDING APPARATUS FOR HIPPOTHERAPY AND METHOD FOR OPERATING SAME

(71) Applicant: Ki Bum LEE, Jeolabuk-do (KR)

(72) Inventor: Ki Bum LEE, Jeolabuk-do (KR)

(21) Appl. No.: 15/538,458

(22) PCT Filed: Dec. 17, 2015
(86) PCT No.: PCT/KR2015/013887

§ 371 Date: Jun. 21, 2017

(30) Foreign Application Priority Data
Dec. 24, 2014 (KR) 10-2014-0189093

(51) Int. Cl. A61B 5/0476; G06Q 50/22; A61H 1/00; A63B 69/04; A61M 21/00

(52) U.S. Cl. CPC A61B 5/0476; A61H 1/005; A63B 69/04; G06Q 50/22; A61H 1/001; A61M 2021/0005

(57) ABSTRACT

A rehabilitation system using an artificially intelligent horseback-riding apparatus for hippotherapy, the system comprising: an artificially intelligent horseback-riding apparatus which is ridable; a smartphone which can communicate with a cloud server and has installed an application for controlling the operation state of the artificially intelligent horseback-riding apparatus and operating the horseback-riding apparatus; a diagnostic device which is for diagnosing the state of a patient by means of a brain wave signal of the patient; an operating unit which is for operating the artificially intelligent horseback-riding apparatus; and a cloud server which is for storing, in the smartphone, treatment program and data, necessary for a patient treatment, and for transmitting same.

### BACKGROUND

The present invention relates to hippotherapy and, more particularly, to a rehabilitation system using an artificial intelligence horse for hippotherapy, which treats disabled persons who require cognitive, physical and emotional rehabilitation using an artificial intelligence horse designed for hippotherapy and an operating method of the same.

### BACKGROUND ART

Hippotherapy has been known to have an excellent effect in neuropsychiatry and disease treatment, such as physical, cognitive and emotional (depression and HDAD) diseases, in particular.

In relation to an exercise using such horseback-riding, there is Korean Utility Model Application No. 20-2014-0003218 (laid open on May 30, 2014) “horseback-riding machine.”

The design relates to an exercise machine which protects the health and safety of a rider against unnecessary vibration while minimizing damage to motor lifespan and a mechanical apparatus by absorbing invalid kinetic energy attributable to a sudden change in the exercise position by associating a motor and a shock absorber in series, which has an improved feeling of horseback-riding, and which does not have a part failure and noise.

However, the conventional technology has a horseback-riding exercise as its object, allows a user to simply have a horseback-riding exercise effect, and was problematic in that the diagnosis and treatment of a psychological state are impossible using the exercise machine.

### SUMMARY OF THE INVENTION

The present invention has a different dimension from the conventional technology and has an object to provide a rehabilitation system using an artificial intelligence horse for hippotherapy, which has an object to treat a patient in a hospital, and an operating method of the same.

A rehabilitation system using an artificial intelligence horse for hippotherapy according to the present invention for achieving the object includes an artificial intelligence horse apparatus capable of horseback-riding, a smartphone which is capable of communication with a cloud server and on which an operating state of the artificial intelligence horse apparatus and an application for driving a horse have been mounted, a diagnostic apparatus which diagnoses the state of a patient through the brainwave signal of the patient, an actuator for driving the artificial intelligence horse apparatus, and the cloud server responsible for a storage and transmission of a treatment program and data necessary for patient treatment to the smartphone. A plurality of application programs which issue a command to the artificial intelligence horse apparatus depending on the state and symptom of the patient is installed on the smartphone, an application which senses the measured values of various sensors including a compass sensor and a gyro sensor is embedded in the smartphone, the diagnostic apparatus including an electroencephalograph for diagnosing the state of the patient is connected to the smartphone, and the smartphone communicates driving data transmitted by the cloud server with an interface board which controls the actuator for the driving of the artificial intelligence horse apparatus through the application program.

Furthermore, the application program temporarily stores and executes a program and data necessary for treatment, transmitted by the cloud server, and sends various diagnostic and treatment data to the cloud server after a treatment program is terminated.

Furthermore, the application program inputs identification code for the patient directly or externally, selects each treatment program according to the state of the patient, and drives the artificial intelligence horse apparatus.

Furthermore, a remote controller for inputting the state of the patient externally is further included.

Furthermore, the application program sends the patient's intention for control and data sensed by various sensors and sensors for patient diagnosis, such as electroencephalograph, which are mounted on the smartphone to the cloud server. The cloud server diagnoses the name and importance of a disease of the patient by analyzing patient data using an analysis algorithm, and guides and stores a prescription program.

Furthermore, the age, sex, body conditions, treatment-elapsed data and treatment program of the patient have been stored in the cloud server. The cloud server is configured to include a communication unit including the Internet and an LTE network, which sends a treatment program performed by the artificial intelligence horse apparatus to the outside and receives data necessary for a treatment program from the outside.

Furthermore, in an operating method of a rehabilitation system using an artificial intelligence horse for hippotherapy according to the present invention for achieving the object, an operating method of an artificial intelligence horse for hippotherapy includes the steps of storing patient information, including the name and age of a patient, required rehabilitation contents, a model or identification information of an artificial intelligence horse being used by the patient, and the on-going situation of a rehabilitation exercise program performed by a member, in the cloud server; guiding a proper rehabilitation program based on patient information selected based on the results of the analysis of a database accumulated and updated in the cloud server; driving the artificial intelligence horse in response to a command from a remote controller, outputting video and voice guidance and a treatment program to 3-D image data, and performing, by the patient, a rehabilitation exercise; and sending the result data to the cloud server when the treatment program is terminated.

Furthermore, the 3-D image data outputs or more images of a trail, a road, a woodland path and the seashore.

Furthermore, the 3-D image data outputs guidance voice, guidance music and road map data necessary for hippotherapy in an image form.

Furthermore, the artificial intelligence horse for hippotherapy is rented with a specific installation fee and a deposit and data access fees are claimed to create income.

### Advantageous Effects

In accordance with the present invention, the following advantages are present compared to traditional hippotherapy using a living horse.

First, there can be provided the rehabilitation system using an artificial intelligence horse for hippotherapy and the operating method of the same having advantages of being safe and almost not having a maintenance cost and space restraints because a trainer does not need to train a horse for treatment and there is no danger of a dangerous behavior that is totally unexpected.

Second, proper prescription according to the treatment state of a patient can continue to be changed through patient treatment data stored in the cloud server.

### BRIEF DESCRIPTION OF DRAWINGS

FIG. 1 shows a schematic configuration for illustrating a rehabilitation system using an artificial intelligence horse for hippotherapy according to the present invention.

FIG. 2 is a diagram schematically showing an artificial intelligence horse for hippotherapy.

FIG. 3 is a flowchart for illustrating an operating method of an artificial intelligence horse for hippotherapy according to the present invention.

Description of reference numerals of
principal elements in the drawings

| 10: artificial intelligent horseback-riding apparatus | |
|---|---|
| 20: sensor unit | 30: actuator |
| 40: communication unit | 50: cloud server |
| 60: diagnostic apparatus | |

### DETAILED DESCRIPTION OF THE INVENTION

An artificial intelligence horse apparatus capable of horseback-riding, a smartphone which is capable of communication with a cloud server and on which an operating state of the artificial intelligence horse apparatus and an application for driving a horse have been mounted, a diagnostic apparatus which diagnoses the state of a patient through the brainwave signal of the patient, an actuator for driving the artificial intelligence horse apparatus, and the cloud server responsible for a storage and transmission of a treatment program and data necessary for patient treatment to the smartphone are included. A plurality of application programs which issue a command to the artificial intelligence horse apparatus depending on the state and symptom of the patient is installed on the smartphone, an application which senses the measured values of various sensors including a compass sensor and a gyro sensor is embedded in the smartphone, the diagnostic apparatus including an electroencephalograph for diagnosing the state of the patient is connected to the smartphone, and the smartphone communicates driving data transmitted by the cloud server with an interface board which controls the actuator for the driving of the artificial intelligence horse apparatus through the application program.

### MODE FOR INVENTION

A preferred embodiment of the present invention is described in detail below with reference to the accompanying drawings.

Furthermore, common terms now widely used have been selected as terms used in the present invention, but in a specific case, a term randomly selected by the applicant is used. In this case, it is to be noted that the present invention should be understood as the meaning inherent in the term not the name of the term simply because the meaning of the term has been clearly described in the description part of a corresponding invention.

Furthermore, in describing the embodiments, a description of contents that have been well known in the art to which the present invention pertains and are not directly related to the present invention is omitted in order to deliver the gist of the present invention clearer by omitting an unnecessary description.

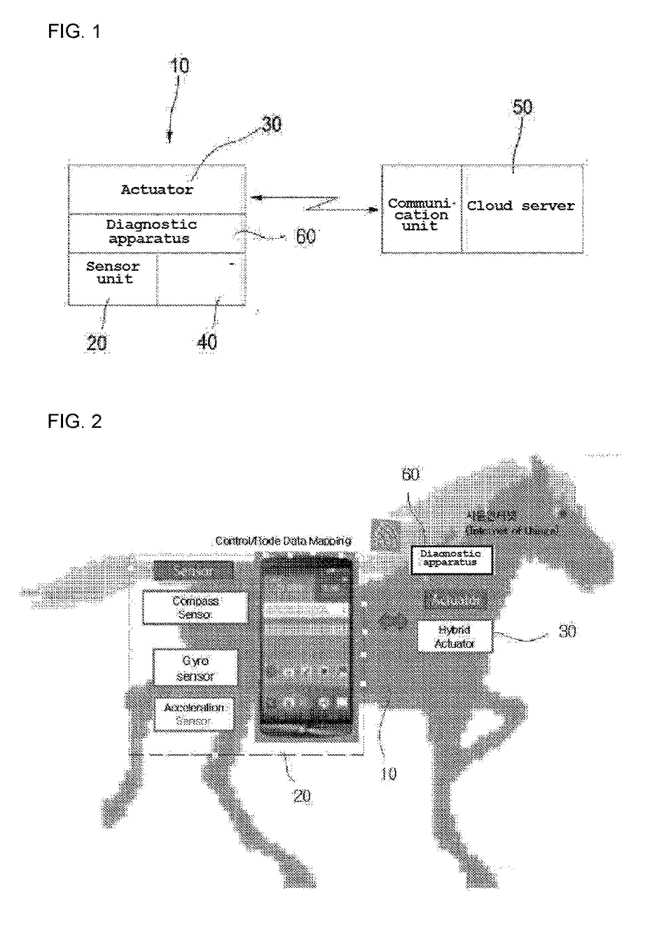

FIG. 1 shows a schematic configuration for illustrating a rehabilitation system using an artificial intelligent horse for hippotherapy according to the present invention. FIG. 2 is a diagram schematically showing the artificial intelligence horse for hippotherapy shown in FIG. 1.

The rehabilitation system using an artificial intelligence horse for hippotherapy according to the present invention includes an artificial intelligent horseback-riding apparatus 10, a sensor unit 20, an actuator 30, a diagnostic apparatus (EEG) 60, a communication unit 40 and a cloud server 50 as shown in FIGS. 1 and 2.

In this case, the artificial intelligent horseback-riding apparatus 10 is a mechanical and device element necessary for rehabilitation and does not need to be specifically limited if it is capable of horseback-riding.

For example, a saddle on which a person can sit, a handle, etc. may be configured in the apparatus.

Bluetooth, etc. capable of communication with a smartphone is applied to the EEG. A plurality of programs that issue commands to the artificial intelligent horseback-riding apparatus depending on the state and symptom of a patient is installed on the smartphone. Applications for sensing the measured values of a compass sensor, a gyro sensor and an acceleration sensor embedded in the smartphone have been embedded in the EEG.

The actuator 30 is for driving a treatment program transmitted to the smartphone in the artificial intelligent horseback-riding apparatus 10, and performs conversion into a mechanical exercise using electricity, a hydraulic cylinder, a hydraulic motor, etc. through an interface board and reproduces a motion of a previously programmed actual horse in the artificial intelligent horseback-riding apparatus 10.

The cloud server 50 sends the control program of the artificial intelligence horse apparatus 10 through communication with a smart device, collects and analyzes treatment data, and databases the data.

Accordingly, it is preferred that the communication unit is configured to provide common wired/wireless Internet, LTE and Bluetooth communication, etc.

Meanwhile, a control program necessary for control of the artificial intelligence horse, such as normal steps, quick steps, a run and a full pace, has been programmed into an application program in the smartphone, that is, an example of a smart device applied to the present invention. In the application program, identification code for a patient is input directly or externally, a treatment program according to the state of the patient is selected, and the artificial intelligence horse apparatus 10 is driven.

The artificial intelligence horse apparatus 10 may be driven by further including a remote controller for inputting the state of a patient externally.

The application program sends data sensed by each of the sensors to the cloud server 50. The cloud server 50 classifies the data into identification code unique to a patient and stores the identification code. Furthermore, the application program temporarily stores and executes a program and data necessary for treatment, transmitted by the cloud server 50, and sends various diagnostic and treatment data to the cloud server 50 after a treatment program is terminated.

Furthermore, the application program inputs identification code for a patient directly or externally, selects each treatment program according to the state of the patient, and drives the artificial intelligence horse apparatus 10. The application program may be configured to further include a remote controller for inputting identification code or a state for a patient externally.

Furthermore, the application program sends a patient's intention for control and data sensed by various sensors and sensors for patient diagnosis, such as electroencephalograph, which are mounted on the smartphone to the cloud server 50.

The cloud server 50 receives the transmitted data, diagnoses the name and importance of a disease of the patient by analyzing the patient data using an analysis algorithm, stores the diagnosed results, and guides a prescription program. In this case, the age, sex, body conditions, treatment-elapsed data and treatment program of the patient have been stored in the cloud server 50. Treatment programs stored in the cloud server 50 include an algorithm which senses and analyzes a reaction from a patient after the artificial intelligence horse apparatus 10 rejects the patient's intention for control.

Furthermore, the cloud server 10 stores the treatment data of a patient received through the application program, analyzes and stores the analyzed and stored data, updates patient information, and databases the updated patient information.

The cloud server 50 applied to the present invention as described above is configured to include the communication unit which sets the age, sex, treatment program and condition of a patient, sends data for a treatment program performed by the artificial intelligence horse apparatus 10 to the outside, and receives data necessary for a treatment program from the outside. In this case, the outside refers to the smartphone mounted on the artificial intelligence horse apparatus 10. In this case, the communication unit of the smartphone may be used as the communication unit 40.

Furthermore, the cloud server 50 applied to the present invention stores diagnostic information, such as the EEG data of a patient received through the application program, the executed prescription data of the artificial intelligence horse apparatus, and the treatment result report data of a medical team, accumulates and updates patient information, and codes and classifies the updated patient information.

Meanwhile, a smartphone application of the present invention confirms the state of a patient who operates in conjunction with the exercise of the artificial intelligence horse apparatus 10, controls a motion, maps it to map data, and sends the map data to a 3-D image apparatus. The 3-D image apparatus includes a 3-D goggle and 3-D monitor connected to a cap for horseback-riding so that the 3-D goggle and 3-D monitor connected to the cap for horseback-riding can display an image. Common wired/wireless Internet or LTE and Bluetooth communication may be used for such transmission.

The present invention extracts the EEG data of a patient who rides or the feature information of the artificial intelligence horse apparatus 10 from information obtained through the sensor unit 20 and the diagnostic apparatus 60, confirms an intention and state of the patient, and databases them.

Meanwhile, it is preferred that 3-D image data includes images of a trail, a road, a woodland path, the seashore, etc. Guidance voice, guidance music and road map data necessary for hippotherapy are output in an image form.

Such hippotherapy is one field of the physical therapy that treats a patient using a horse for horseback-riding and has been proved to have an excellent therapy effect, but a horse for horseback-riding has not been activated due to a high cost and space restraint if it is directly used.

If such an artificial intelligence horse is used, however, medical personnel can perform patient treatment more safely and easily and can a burden of a patient can be reduced.

Such hippotherapy may be defined as “all of activities in which a human being and a horse act together in order to provide cognitive, physical, emotional and social peace to disabled persons.”

Horseback-riding effects include diet, an increased bowel function, an aerobic exercise, an increased muscle such as the pelvis and sphincter, increased waist flexibility, lower body training, backbone correction, a strengthened mental power, a rehabilitation function effect, a growth effect, a high oxygenation effect, a blood-sugar reduction effect and so on.

From among them, the rehabilitation function effect includes the rehabilitation therapy effect of mental diseases, such as depression and autism.

Such hippotherapy has effects of ADHD treatment, motivation, improved judgment, depression treatment, improved confidence and improved concentration from a mental aspect, can improve the quality of life, such as the improved communication ability and the improved social adaptation ability from a social aspect, and has effects of a joint motion, a sense of balance, a coordination ability, a muscular strength, endurance, strengthened muscle, and the normalization of muscle tension from a physical aspect.

In particular, if an unbroken horse is used, there are problems in that much time and cost are required to train a living horse for hippotherapy, there is a danger that a patient may be injured due to an unexpected behavior of a horse that is a life, and there are a space restraint and the shortage of manpower for training and breeding a horse.

In contrast, an artificial intelligence horse for hippotherapy has advantages in that it does not require time and cost for training for treatment by a trainer and does not have a danger of a dangerous and unexpected behavior and it is safe and almost does not have a maintenance cost and space restraint.

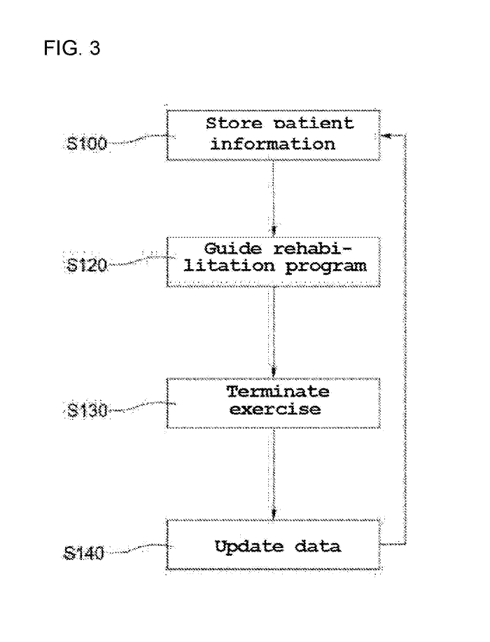

FIG. 3 is a flowchart for illustrating an operating method of the artificial intelligence horse for hippotherapy according to the present invention.

As shown in FIG. 3, in the operating method of an artificial intelligence horse for hippotherapy according to the present invention, patient information is stored (S100).

The patient information includes a name, an age, required rehabilitation contents, a model or identification information of an artificial intelligence horse used by the patient, the on-going situation of a rehabilitation exercise program performed by a member and so on.

Next, a suitable rehabilitation program may be guided based on the patient information (S110).

In this case, the rehabilitation program is selected based on the results of the analysis of a database accumulated and updated in the cloud server 50.

Next, the artificial intelligence horse may be driven in response to a command by a remote controller, etc., and thus the patient performs a rehabilitation exercise (S120). In this case, video and voice guidance, a treatment program, music, etc. may be output to 3-D image data. In the 3-D image data, one or more images of a trail, a road, a woodland path and the seashore may be output as road map data. The 3-D image data may be output through the 3-D image apparatus. The 3-D image apparatus may include a 3-D goggle and 3-D monitor connected to a cap for horseback-riding, so that the 3-D goggle and 3-D monitor connected to the cap for horseback-riding may output an image.

Meanwhile the artificial intelligence horse for hippotherapy may be rented with a specific installation fee and a deposit, and such data access fees may be claimed to create income.

Although the present invention has been described with reference to the accompanying drawings, this is only one of various embodiments including the gist of the present invention and has its object to allow a person having ordinary skill in the art to easily practice the embodiments. Accordingly, it is evident that the present invention is not limited to only the described embodiments. Accordingly, the range of protection of the present invention should be interpreted based on the following appended claims. Accordingly, all of technological spirits within equivalents thereof may be included in the scope of right of the present invention by changes, substitutions, replacements, etc. within a range that does not depart from the gist of the present invention. Furthermore, it is evident that some elements in the drawings have been illustrated to provide a clearer description and the size thereof has been more enlarged or reduced than its actual size.

In accordance with the present invention, the following advantages are present compared to traditional hippotherapy using a living horse. First, there can be provided the rehabilitation system using an artificial intelligence horse for hippotherapy and the operating method of the same having advantages of being safe and almost not having a maintenance cost and space restraints because a trainer does not need to train a horse for treatment and there is no danger of a dangerous behavior that is totally unexpected. Second, proper prescription according to the treatment state of a patient can continue to be changed through patient treatment data stored in the cloud server. Accordingly, the present invention is an invention having a very high industrial applicability.

1. A rehabilitation system using an artificial intelligence horse for hippotherapy, the system comprising:
an artificial intelligence horse apparatus capable of horseback-riding;
a smartphone which is capable of communication with a cloud server and on which an operating state of the artificial intelligence horse apparatus and an application for driving a horse have been mounted;
a diagnostic apparatus which diagnoses a state of a patient through a brainwave signal of the patient;
an actuator for driving the artificial intelligence horse apparatus; and
the cloud server responsible for a storage and transmission of a treatment program and data necessary for patient treatment to the smartphone,
wherein a plurality of application programs which issue a command to the artificial intelligence horse apparatus depending on the state and symptom of the patient is installed on the smartphone, an application which senses measured values of various sensors comprising a compass sensor and a gyro sensor is embedded in the smartphone, the diagnostic apparatus comprising an electroencephalograph for diagnosing the state of the patient is connected to the smartphone, and the smartphone communicates driving data transmitted by the cloud server with an interface board which controls the actuator for the driving of the artificial intelligence horse apparatus through the application program.

2. The rehabilitation system of claim 1, wherein horseback-riding steps regarding normal steps, quick steps, a run and a full pace and treatment programs have been programmed in the application program.

3. The rehabilitation system of claim 1, wherein:
the application program sends patient state data sensed by the diagnostic apparatus comprising the electroencephalograph to the cloud server, and
the cloud server classifies the patient state data into identification code unique to the patient and stores the identification code.

4. The rehabilitation system of claim 1, wherein:
a program for inputting and selecting an age, sex and body conditions of the patient and an effect to be achieved and treating the patient has been stored in the cloud server, and
the cloud server is configured to comprise a communication unit which sends data for a treatment program performed by the artificial intelligence horse apparatus to the smartphone and receives data necessary for a treatment program from the smartphone.

5. The rehabilitation system of claim 1, wherein the cloud server confirms a state of the patient operating in conjunction an exercise of the artificial intelligence horse apparatus through communication with the smartphone, controls a motion, maps the motion to 3-D image data, and sends the 3-D image data to a display unit of the smartphone.

6. The rehabilitation system of claim 1, wherein:
one or more of a treatment program, 3-D road map data, an artificial intelligence horse apparatus driving program, a patient treatment data analysis algorithm, patient data, applied treatment program information, an amount of data used, and a fee charge system according to a use program have been stored in the cloud server, and
the information is connected to the smartphone mounted on the artificial intelligence horse apparatus in a wired or wireless manner through the Internet and an LTE network.
